(19) United States
(12) Patent Application Publication
(10) Pub. No.: US 2024/0065891 A1
(43) Pub. Date: Feb. 29, 2024

(54) BIOLOGICAL FLUID DRAINAGE DEVICES AND METHODS

(71) Applicant: W. L. Gore & Associates, Inc., Newark, DE (US)

(72) Inventors: Jesse R. McAlister, Newark, DE (US); Jeffrey C. Towler, Wilmington, DE (US)

(21) Appl. No.: 18/237,045

(22) Filed: Aug. 23, 2023

Related U.S. Application Data
(60) Provisional application No. 63/401,258, filed on Aug. 26, 2022.

(51) Int. Cl. A61F 9/007

(52) U.S. Cl. CPC A61F 9/00781

(57) ABSTRACT

A drainage device for draining a biological fluid from an eye to a tissue external to the eye is disclosed. The drainage device is implantable at least in part within a tissue of the eye. The drainage device includes: an open collapsible body portion having a first surface with a first porosity and a second surface with a second porosity that is greater than the first porosity, the second surface opposing the first surface; and a conduit having a first end fluidly coupled with the first surface and a second end insertable into the eye to facilitate a drainage of the biological fluid into the conduit. The second surface with the second porosity includes at least one ingrowth surface region for facilitating ingrowth of tissue external to the eye.

### CROSS REFERENCE TO RELATED APPLICATION

This application claims the benefit of U.S. Provisional Application No. 63/401,258, filed Aug. 26, 2022, which is incorporated by reference herein in its entirety for all purposes.

### FIELD

The present disclosure relates generally to apparatuses and methods for draining biological fluid and diverting the fluid to be reabsorbed elsewhere in the body. More specifically, the disclosure relates to apparatuses and methods for draining aqueous humor from an anterior chamber (AC) of a patient's eye such that it may be reabsorbed by the body.

### BACKGROUND

Various medical interventions involve evacuating excess biological fluid from one portion of the body and redirecting it to another location of the body where it may be reabsorbed. In certain instances, this evacuation is achieved via minimally invasive procedures such as endoscopic third ventriculostomy (ETV) and choroid plexus cauterization procedure (CPC). In other instances, this evacuation is performed post-operatively via implantable medical devices, such as a shunt. Proven useful in various medical procedures, shunts of different forms have been employed as treatment for numerous diseases, such as hydrocephalus and glaucoma.

Without treatment, excessive biological fluid may lead to unhealthy pressure buildups. For instance, glaucoma is a progressive eye disease characterized by elevated intraocular pressure. Aqueous humor is a fluid that fills an anterior chamber (AC) of the eye and contributes to intraocular pressure or intraocular fluid pressure. This increase in intraocular pressure is usually caused by an insufficient amount of aqueous humor absorbed by the body. In some cases, the aqueous humor is not absorbed quickly enough or even not absorbed at all, while in other cases, the aqueous humor is additionally or alternatively produced too quickly. Elevated intraocular pressure is associated with gradual and sometimes permanent loss of vision in the affected eye.

Attempts have been made to treat glaucoma, including devices that are relatively bulky, inflexible, uncompliant, and lack secure anchoring capabilities to surrounding tissue resulting in relative movement between the device and tissue. Such movement may result in continued stimulation of the surrounding tissue, causing irritation at the implantation site. Irritation, in turn, may lead to increased chronic inflammatory tissue response, excessive scarring at the device site, and increased risk of device erosion through conjunctiva and endophthalmitis. Scar tissue effectively prevents resorption of aqueous humor without erosion and interferes with device function.

### SUMMARY

Disclosed herein are devices for draining a biological fluid from an eye to a tissue surrounding the eye, as well as methods for forming the device that is implantable at least in part within a tissue of an eye and methods for treating a glaucoma using the device.

According to one example (“Example 1”), a drainage device for draining a biological fluid from an eye to a tissue external to the eye is disclosed. The drainage device is implantable at least in part within a tissue of the eye. The drainage device includes: an open collapsible body portion having a first surface with a first porosity and a second surface with a second porosity that is greater than the first porosity, the second surface opposing the first surface; and a conduit having a first end fluidly coupled with the first surface and a second end insertable into the eye to facilitate a drainage of the biological fluid into the conduit. The second surface with the second porosity includes at least one ingrowth surface region for facilitating ingrowth of tissue external to the eye.

According to another example (“Example 2”) further to Example 1, the first surface with the first porosity is configured to inhibit the ingrowth of the tissue external to the eye.

According to another example (“Example 3”) further to Example or 2, the second surface further includes at least one low-porosity region for inhibiting the ingrowth of the tissue external to the eye.

According to another example (“Example 4”) further to Example 3, the at least one low-porosity region in the second surface defines a third surface opposing the first surface that is discontinuous from the second surface and has a third porosity that is less than the second porosity.

According to another example (“Example 5”) further to Example 4, the third porosity is equal to the first porosity.

According to another example (“Example 6”) further to any one of the preceding Examples, the body portion comprises a plurality of internal regions with porosities that are greater than the first porosity and less than the second porosity.

According to another example (“Example 7”) further to any one of the preceding Examples, the conduit is attached to a periphery of the body portion.

According to another example (“Example 8”) further to any one of Examples 1-6, the conduit is attached to the body portion across at least 50% of a cross-sectional length of the body portion.

According to another example (“Example 9”) further to any one of Examples 1-6, the conduit is attached to the first surface of the body portion at an attachment region, and the body portion further comprises an erosion element with a trimmable portion that extends outwardly from the attachment region with respect to the conduit.

According to another example (“Example 10”) further to Example 9, the erosion element is a separate component from the body portion.

According to another example (“Example 11”) further to Example 9, the erosion element is a continuous extension of the body portion.

According to another example (“Example 12”) further to any one of the preceding Examples, the body portion includes: an inner component having a first surface with a first surface porosity and a second surface with a second surface porosity that is greater than the first surface porosity, and an outer component having a first surface with a first surface porosity and a second surface with a second surface porosity that is greater than the first surface porosity. The inner and outer components are disposed such that the first surface of the inner component faces the first surface of the outer component.

According to another example (“Example 13”) further to Example 12, the conduit is disposed between the first surface of the inner component and the first surface of the outer component.

According to another example (“Example 14”) further to Example 12 or 13, the device further includes one or more additional inner components each having a first surface with a first surface porosity and a second surface with a second surface porosity that is greater than the first surface porosity. The additional inner components are attached to the outer component such that each of the first surfaces of the additional inner components faces the first surface of the outer component.

According to another example (“Example 15”) further to any one of the preceding Examples, the device further includes a support structure attached to at least a portion of the body portion.

According to another example (“Example 16”) further to Example 15, the conduit is attached to a portion of the support structure.

According to another example (“Example 17”) further to Example 15 or 16, the support structure comprises one or more thermoplastic components.

According to another example (“Example 18”) further to Example 17, one or more channels are formed between two of the thermoplastic components through which the biological fluid is configured to pass.

According to another example (“Example 19”) further to any one of Examples 15-18 that is further to Example 12, the support structure is disposed between the first surface of the inner component and the first surface of the outer component.

The foregoing Examples are just that, and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1A is a schematic drawing of a cross-sectional view of a drainage device according to embodiments disclosed herein;

FIG. 1B is an SEM image of a portion of a cross-sectional view of the drainage device according to embodiments disclosed herein (the image is to the scale shown in the image);

FIG. 1C is a schematic drawing of a cross-sectional view of an eye when the drainage device is at least partially implanted according to embodiments disclosed herein;

FIG. 1D is a microscopic image of a tissue slide stained with a histological stain agent showing a cross-section of the drainage device according to embodiments disclosed herein (the image is to the scale shown in the image);

FIG. 1E is a schematic drawing of a partial view of a multilumen fluid conduit according to embodiments disclosed herein;

FIGS. 2A and 2B are schematic drawings of a bottom view of drainage devices according to embodiments disclosed herein;

FIG. 3 is a schematic drawing of a cross-sectional view of a drainage device according to embodiments disclosed herein;

FIGS. 4 through 6 are schematic drawings of an angled view of drainage devices according to embodiments disclosed herein;

FIG. 7A is a schematic drawing of a bottom view of a drainage device according to embodiments disclosed herein;

FIG. 7B is a schematic drawing of a top view of the drainage device of FIG. 7A;

FIG. 7C is a schematic drawing of a cross-sectional view of the drainage device of FIG. 7B as cut across line C-C;

FIG. 8A is a schematic drawing of a bottom view of a drainage device according to embodiments disclosed herein;

FIG. 8B is a schematic drawing of a top view of the drainage device of FIG. 8A;

FIG. 8C is a schematic drawing of a cross-sectional view of the drainage device of FIG. 8B as cut across line C-C;

FIG. 9A is a schematic drawing of a bottom view of a drainage device according to embodiments disclosed herein;

FIG. 9B is a schematic drawing of a top view of the drainage device of FIG. 9A;

FIG. 9C is a schematic drawing of a cross-sectional view of the drainage device of FIG. 9B as cut across line C-C;

FIG. 10A is a schematic drawing of a bottom view of a drainage device according to embodiments disclosed herein;

FIG. 10B is a schematic drawing of a top view of the drainage device of FIG. 10A;

FIG. 10C is a schematic drawing of a cross-sectional view of the drainage device of FIG. 10B as cut across line C-C;

FIG. 11A is a schematic drawing of a bottom view of a drainage device according to embodiments disclosed herein;

FIG. 11B is a schematic drawing of a top view of the drainage device of FIG. 11A;

FIG. 11C is a schematic drawing of a cross-sectional view of the drainage device of FIG. 11B as cut across line C-C;

FIGS. 11D and 11E are schematic drawings of a bottom view of the drainage device of FIG. 11A with two different attachment locations according to embodiments disclosed herein;

FIG. 11F is a photograph of a top view of a drainage device according to embodiments disclosed herein;

FIG. 11G is a photograph of a bottom view of the drainage device of FIG. 11F;

FIG. 12A is a schematic drawing of a bottom view of a drainage device according to embodiments disclosed herein;

FIG. 12B is a schematic drawing of a top view of the drainage device of FIG. 12A;

FIG. 12C is a schematic drawing of a cross-sectional view of the drainage device of FIG. 12B as cut across line C-C;

FIG. 13A is a schematic drawing of a bottom view of a drainage device according to embodiments disclosed herein;

FIG. 13B is a schematic drawing of a top view of the drainage device of FIG. 13A;

FIG. 13C is a schematic drawing of a cross-sectional view of the drainage device of FIG. 13B as cut across line C-C;

FIG. 14A is a schematic drawing of a bottom view of a drainage device according to embodiments disclosed herein;

FIG. 14B is a schematic drawing of a top view of the drainage device of FIG. 14A;

FIG. 14C is a schematic drawing of a cross-sectional view of the drainage device of FIG. 14B as cut across line C-C;

FIG. 15A (prior art) is a photograph of a top view of an Ahmed glaucoma valve modified with a polyethylene shell and implementing solid plates as known in the art;

FIG. 15B (prior art) is a photograph of a side view of the prior-art glaucoma valve of FIG. 15A when a portion of the solid plates are removed to show the reservoir located therein; and

FIG. 15C (prior art) is an SEM image of a portion of a surface of the solid plate used in the prior-art glaucoma valve of FIG. 15A (the image is to the scale shown in the image).

It should be understood that the drawings and replicas of the photographs are not necessarily to scale, unless indicated otherwise. In certain instances, details that are not necessary for an understanding of the disclosure or that render other details difficult to perceive may have been omitted. It should be understood, of course, that the disclosure is not necessarily limited to the particular examples or embodiments illustrated or depicted herein.

### DETAILED DESCRIPTION

### Definitions and Terminology

This disclosure is not meant to be read in a restrictive manner. For example, the terminology used in the application should be read broadly in the context of the meaning those in the field would attribute such terminology.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X₁-Xₙ, Y₁-Yₘ, and Z₁-Zₒ, the phrase is intended to refer to a single element selected from X, Y, and Z, a combination of elements selected from the same class (e.g., X₁ and X₂) as well as a combination of elements selected from two or more classes (e.g., Y₁ and Zₒ).

It should be understood that every maximum numerical limitation given throughout this disclosure is deemed to include each and every lower numerical limitation as an alternative, as if such lower numerical limitations were expressly written herein. Every minimum numerical limitation given throughout this disclosure is deemed to include each and every higher numerical limitation as an alternative, as if such higher numerical limitations were expressly written herein. Every numerical range given throughout this disclosure is deemed to include each and every narrower numerical range that falls within such broader numerical range, as if such narrower numerical ranges were all expressly written herein.

Before any embodiments of the disclosure are explained in detail, it is to be understood that the disclosure is not limited in its application to the details of construction and the arrangement of components set forth in the following description or illustrated in the following drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

The term “fibril” as used herein describes an elongated piece of material such as a polymer, where the length and width are substantially different from each other. For example, a fibril may resemble a piece of string or fiber, where the width (or thickness) is much shorter or smaller than the length.

The term “node” as used herein describes a connection point of at least two fibrils, where the connection may be defined as a location where the two fibrils come into contact with each other, permanently or temporarily. In some examples, a node may also be used to describe a larger volume of polymer than a fibril and where a fibril originates or terminates with no clear continuation of the same fibril through the node. In some examples, a node has a greater width but a smaller length than the fibril.

As used herein, “nodes” and “fibrils” may be used to describe objects that are usually, but not necessarily, connected or interconnected, and have a microscopic size, for example. A “microscopic” object may be defined as an object with at least one dimension (width, length, or height) that is substantially small such that the object or the detail of the object is not visible to the naked eye or difficult, if not impossible, to observe without the aid of a microscope (including but not limited to a scanning electron microscope or SEM, for example) or any suitable type of magnification device.

### Description of Various Embodiments

Various embodiments address a drainage device which can be implanted in the eye to drain such fluid from the AC of an eye and accommodate for means of avoiding or preventing hypotony. However, many prior-art drainage devices merely define a lumen to redirect the fluid from the AC to a different location with a lower pressure than the AC and therefore do not include any component which redirects the flow of the fluid after it leaves the AC. That is, in contrast to various embodiments described herein, such drainage devices do not include a component which accommodates fluid when it arrives at the location with lower pressure than the AC.

Furthermore, in contrast to some embodiments described herein, some prior-art drainage devices only provide an initial flow restriction during implantation of the drainage device. For example, devices using a dissolvable plug section provide an initial outflow resistance to avoid early low post-operation intraocular pressures and hypotony, and subsequently increase in flow over time by lessening the outflow resistance in order to compensate for the rising biological outflow resistance after the operation is complete, but do not provide a long-term means of avoiding or preventing hypotony within the eye.

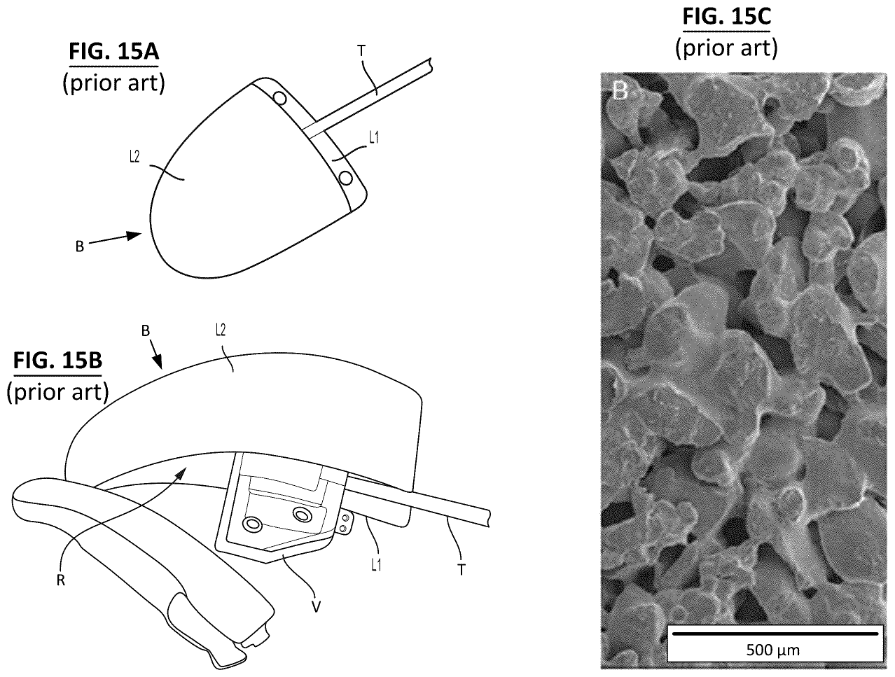

Other prior-art drainage devices may implement solid plates, such as those made from silicone as shown in FIGS. 15A through 15C, to receive fluid from the AC of the eye and store it inside an internal chamber formed between the plates. The device shown in FIGS. 15A and 15B is an Ahmed Glaucoma Value model M4 (New World Medical, Inc., Rancho Cucamonga, CA) with a polyethylene shell to reduce the fibrotic reaction around the drainage plate compared with the S2 and FP7 models in patients with glaucoma, as previously disclosed in Kim J, Allingham R R, Hall J, et al. “Clinical experience with a novel glaucoma drainage implant.” Journal of Glaucoma. 2014 February; 23(2):e91-7. DOI: 10.1097/ijg.0b013e3182955d73. PM ID: 23689073. The prior-art device is made of a tube (T) installed between two layers (L1 and L2) of solid material such as porous polyethylene shells (e.g., Medpor) that follow the curvature of the eye, and between the layers L1 and L2 is defined a reservoir (R) into which the tube T directs the fluid from the eye. As shown in the prior-art device of FIG. 15C, which is scaled such that the black bar on the bottom of the figure represents 500 μm, the surface of the layers L1 and L2 include pores to improve tissue integration, such that the adjacent tissue can be integrated into the polyethylene shell surrounding the drainage device, as well as to ensure that fluid stored inside the reservoir R can be released back to the surrounding environment in order to prevent the shell from applying undue stress on the surrounding tissue.

However, in contrast to various embodiments described herein, implanting a solid or rigid piece of material, such as those shown in FIGS. 15A to 15C, (that is, a material lacking in flexibility and thinness) inside the eye increases the stress applied on the eye, especially within the conjunctival tissue of the eye, when subjected to pressure from the AC, which may not only lead to discomfort but also other complications that may arise from extended pressure increase within such a sensitive area of the eye.

Persons skilled in the art will readily appreciate that various aspects of the present disclosure may be realized by any number of methods and apparatuses configured to perform the intended functions. It should also be noted that the accompanying drawing figures referred to herein are not necessarily drawn to scale (unless indicated otherwise), but may be exaggerated to illustrate various aspects of the present disclosure, and in that regard, the drawing figures should not be construed as limiting.

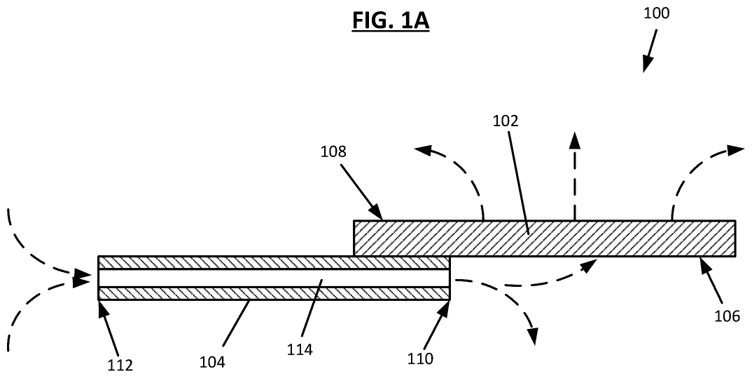

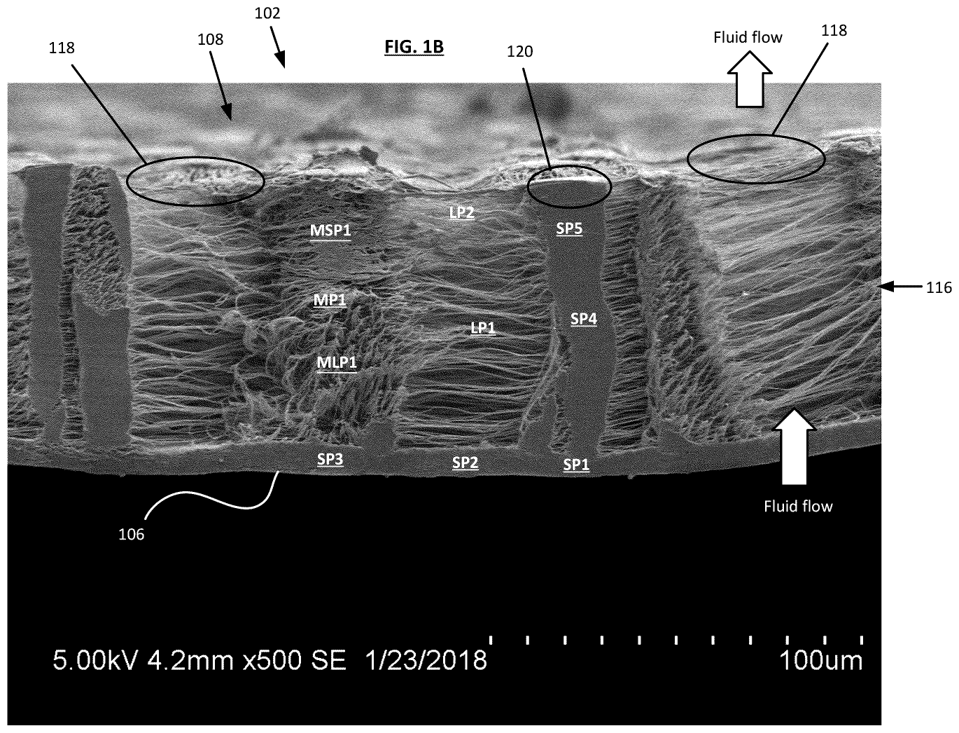

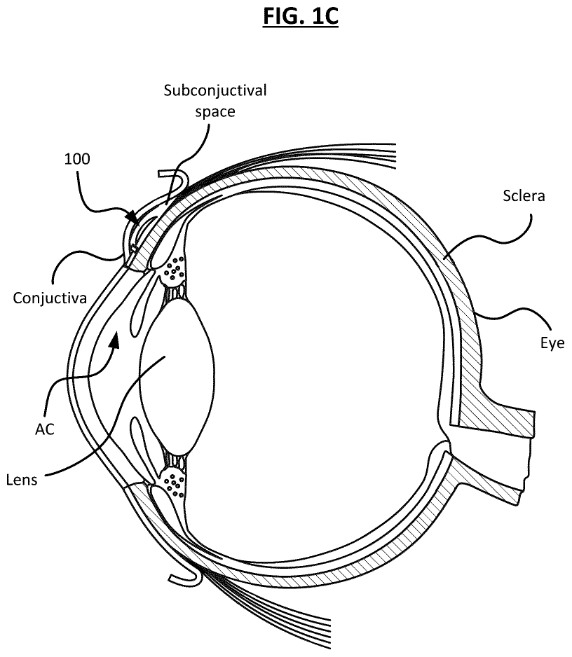

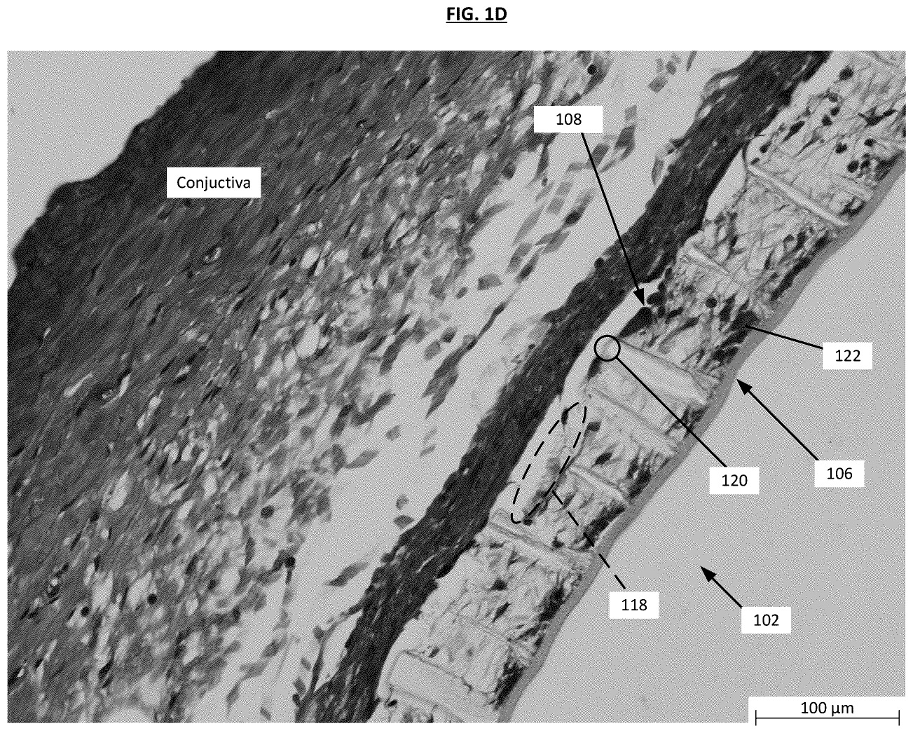

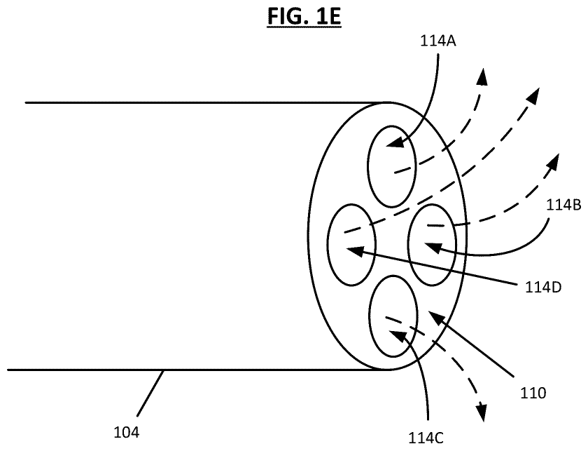

Various aspects of the present disclosure relate to drainage devices and methods for biological fluids. For example, the present disclosure addresses devices and methods for draining aqueous humor from an anterior chamber (AC) of an eye (FIG. 1C) of a patient so that the aqueous humor may be resorbed by the body elsewhere. FIG. 1C is an illustration of an eye with a subconjunctival space between a conjunctiva and a sclera of the eye. Implanted within the eye is a drainage system with a drainage device 100 in accordance with principles of the present disclosure. In an aspect of the present disclosure, a mechanism is provided for reabsorption of aqueous humor that has been expelled from the AC of the eye to reduce or otherwise stabilize intraocular pressure. One skilled in the art, however, will appreciate that aspects of the present disclosure are useful in other applications where drainage of biological fluid to be redirected in the body is desired. In some examples, the target implantation locations may include subconjunctival and/or sub-Tenon locations of the eye. In some examples, the device 100 may be at least partially subconjunctivally implanted and at least partially suprachoroidally implanted (e.g., posterior of the pars plana of the eye), as suitable for the intended treatment.

Different embodiments and examples of drainage device 100 for treating glaucoma are disclosed herein. For example, FIG. 1A shows an example of the device 100. As shown, the device 100 is an implantable device which, when implanted at least in part within the tissue of an eye, can be used for draining biological fluid from the eye. In some examples, the fluid may be drained to a tissue surrounding the eye or to a tissue external to the eye. The device 100 includes an open collapsible body portion 102 and a fluid conduit 104. The body portion 102 has a first surface 106 with a first porosity as well as a second surface 108 with a second porosity that is greater than the first porosity. The second surface 108 opposes (or is disposed in a location of the body portion 102 that is opposite from the location of) the first surface 106. The body portion 102 is “open” because there is no space or chamber enclosed therein, thereby lacking the chamber or “reservoir” as shown in the prior-art device of FIG. 15B.

In some examples, thickness of the body portion 102 may range from about 25 μm to about 30 μm, about 30 μm to about 40 μm, about 40 μm to about 50 μm, about 50 μm to about 60 μm, from about 60 μm to about 70 μm, from about 70 μm to about 80 μm, from about 80 μm to about 90 μm, from about 90 μm to about 100 μm, from about 10 μm to about 150 μm, from about 150 μm to about 200 μm, from about 200 μm to about 250 μm, from about 250 μm to about 300 μm, from about 300 μm to about 350 μm, from about 350 μm to about 400 μm, from about 400 μm to about 450 μm, from about 450 μm to about 500 μm, or any other suitable value or range therebetween and/or combination of ranges thereof. In some examples, the body portion 102 may have a diameter in the range of 5 mm to 15 mm, such as 10 mm for example. In some embodiments, the body portion 102 may be ovular and include a major dimension (e.g., along a major axis of the ellipse) of up to about 30 mm and corresponding minor dimension (e.g., along a minor axis of the ellipse) of up to about 10 mm. As discussed above, given differing anatomies of the human body, the body portion 102 may exceed such dimensions (e.g., 10 mm, 15 mm, and 30 mm) provided that the size does not substantially interfere with normal eye functioning (e.g., pivoting and blinking) or substantially reduce the flexibility of the aqueous humor diffusion member undesirable relative movement occurs between the drainage device 100 and the surrounding tissue when implanted, resulting with a likely consequence of tissue irritation, foreign body tissue response, and/or excessive scar formation. Likewise, the body portion 102 may have a diameter of less than 5 mm, 3 mm, or even less than 3 mm provided that the body portion 102 is operable to accommodate a sufficient degree of evacuated aqueous humor and is operable to facilitate the reabsorption of aqueous humor to constitute an effective treatment for the patient.

The conduit 104 has a first end 110 and a second end 112. The conduit 104 may be a tube or any suitable construct that allows passage of fluid therethrough. The first end 110 is fluidly coupled with the first surface 106, and the second end 112 is insertable into the eye of the patient to facilitate a drainage of the biological fluid into the conduit 104. Throughout the figures disclosed herein, an arrow with a broken line attached thereto represents the general directions in which fluid may flow with respect to the components of the device 100. For example, in FIG. 1A, fluid may flow into a lumen 114 of the conduit 104 via the second end 112, and after passing through the lumen 114, exit via the first end 110. Thereafter, the fluid may flow away from the body portion 102 or toward the body portion 102 such that the fluid enters the body portion 102 from the first surface 106, travels through the material of the body portion 102, and leave the body portion 102 from the second surface 108. The second surface 108 with the second porosity also includes at least one ingrowth surface region 118 which facilitates ingrowth of the tissue as illustrated in FIG. 1B.

FIG. 1B shows a microscopic view of a microporous material of the external surface (second surface 108) of the body portion 102 of the device 100 according to some embodiments. The body portion 102 includes a plurality of internal regions 116 between the first surface 106 and the second surface 108, with porosities that are greater than the first porosity and less than the second porosity. Displayed at the bottom of FIG. 1B is: “5.00 kV 4.2 mm×500 SE 1/23/2018,” and the distance between two consecutive lines as shown at the bottom right hand corner represents 10 μm. For example, the microporous material of FIG. 1B may be referred to throughout with reference to a medical implant device or system. As can be appreciated by a person of skill in the art and with reference to FIG. 1B, the microporous aspects and parameters of the microporous material can be defined in a variety of ways. In an application of a microporous material in an ocular device, such as the device 100 described herein, configured for in situ placement in the tissue of the eye to facilitate the drainage of a biological fluid from the eye (as shown by the white arrows labeled “fluid flow” representing the flow direction of the biological fluid with respect to the first surface 106 and the second surface 108), the microporous properties of such a microporous material can be generally characterized by a volumetric porosity value that can be defined as a ratio of a volume of the air or fluid defined by and contained within the microporous material as compared to an overall volume (or total volume) of the microporous material.

In another definition, a volumetric porosity can be defined as a percentage of the microporous material volume that is occupied by non-structural or transient elements such as air or other fluids. For example, a microporous material with an overall volume of 100 mm³ and with 30 mm³ of that volume comprising chambers holding air or a fluid would have a volumetric porosity value of 0.3 because 30% of the volume of the microporous material is empty or transient space that is filled with air or other fluids.

As can be appreciated, two microporous materials can have the same volumetric porosity but differ in the pore sizes presented to the incoming or exiting air or fluid. For example, a first material can a have a small number of large pores distributed over a fixed overall volume and a second material can have a relatively large number of relatively smaller pores distributed over the same fixed volume, and both microporous materials could have the same volumetric porosity if the air/fluid volume of the two materials are the same.

As can be further appreciated, the properties of the microporous materials used in an ocular drainage device can also be defined by the size of the passages passing through the microporous material or similarly defined as a pore size measured where a passage terminates at a surface of the microporous material or measured along a length of a passage within the material. Microporous materials with small pores or passages can impede flow through the material and comparatively large pores or passages can provide an increased pass through of the air or fluid into, out of, or within the microporous material.

As can be still further appreciated, the properties of the microporous material can also be defined by a tortuosity of the passages entering into and passing through the material, with relatively small or large passages presenting impeded fluid pathways due the frequency of turns in the passages or by the placement of obstructions in the fluid pathways. The air/fluid passthrough rates of a microporous material can be managed by controlling or defining any of the above-described characteristics of the material to provide a suitable material for use to facilitate the drainage of a biological fluid from the eye.

For simplicity, the aforementioned characteristics and variables of the microporous material used in the various embodiments and examples described herein can be presented simply as a porosity which can be based on a volumetric porosity, a pore or passage size, or a tortuosity metric. Again, with reference to FIG. 1B, internal portions of the microporous material can have varying porosities (or volumetric porosities, or pore sizes, or tortuosities). The internal portions can extend between an inner surface (first surface 106) and the external surface (second surface 108).

At any of these portions of a body portion 102, the porosity can comparatively range in degree from small pore size (SP), medium-small pore size (MSP), medium pore size (MP), medium-large pore size (MLP), and large pore size (LP). Assuming, for discussion purposes here, that delivery travels along a relatively straight path through a microporous material so as to sequentially engage porosities of the inner surface 106, a uniform internal portion, and the external surface 108, the combined flow resistance can be represented by likewise concatenating their respective porosities. For instance, the inner surface 106 typically has a low porosity throughout (e.g., to resist tissue ingrowth through the body portion 102, and/or to maintain a plane between the device 100 and the sclera), and portions of the interior portions and the external surface 108 can have any of the aforementioned degrees of porosity. Under these circumstances when the internal portion has a medium porosity and, for example, the internal portions have a medium porosity and the external surface 108 has a high porosity, the drainage of a biological fluid from the eye through the microporous material to tissue surrounding the device can be represented as SP-MP-LP. More examples are discussed here below.

Various delivery paths can be present within the microporous material. Relatively linear flow paths may comprise regions SP1-SP4-SP5, for example or SP3-MLP1-MP1-MSP1. Although some flow paths may be relatively straight, there are also flow paths that are nonlinear. For instance, under certain conditions, at least some flow may proceed to flow through areas of increasingly less resistance such as SP1-LP1-LP2 or SP3-MLP1-LP1-LP2. As will be appreciated, the microstructure of the microporous materials may undergo modification processes to obtain certain types of flow through the microstructure. For instance, the microstructure may have relatively uniform layers across layered within the microstructure, or as shown here, have variable portions throughout the thickness of the microporous material.

In some examples, the body portion 102 defines a wall portion thickness extending between the inner surface 106 and the external surface 108. The wall portion thickness can define an internal region of the body portion 102 having a transition porosity that is between a porosity of the low porosity surface (e.g., having smaller pore sizes) of the inner surface 106 and a porosity of the high porosity surface (e.g., having larger pore sizes) of the external surface 108. In addition, or in alternative, the internal region can have an internal region porosity that is equal to porosities of the low porosity surfaces of the inner surface 106 and the external surface 108. In addition, or in alternative, the internal region can have an internal region porosity that is equal to a porosity of the low porosity surface of the inner surface 106. In addition, or in alternative, the internal region can have an internal region porosity that is equal to a porosity of the high porosity surface of the external surface 108.

With reference still to the microporous material shown in FIG. 1B, the fluid pathways may also be impacted by a pressure difference between the fluid in one area (e.g., at the first surface 106) and the fluid in another area (e.g., at the second surface 108). In some examples, the first surface 106 has the first porosity that prevents or inhibits the ingrowth of the tissue. In some examples, the second surface 108 includes at least one low-porosity region 120 for inhibiting the ingrowth of the tissue. The regions capable of inhibiting tissue ingrowth may have relatively small pore sizes, such as SP and/or MSP as labeled, for example.

FIG. 1C shows an exemplary location in which the device 100 may be implanted in the eye. In the example shown, the device 100 is sized and shaped such that it is implantable within a dissected subconjunctival space, such as between a sclera and a conjunctiva of the patient's eye.

As discussed above, in various embodiments, the fluid conduit is a soft and compliant tubular member insertable into the anterior chamber of the eye. In various example surgical approaches, one or more of the fluid conduit and the aqueous humor diffusion member will be advanced or pushed during the implantation procedure. Soft, thin, and compliant tubular structures are generally difficult to advance through tissue. Accordingly, in various embodiments, the glaucoma drainage devices discussed herein may further include a stiffening member such as a support structure that is integrated with the devices. The stiffening member may form an installation assembly having column strength in excess of the column strength of the fluid conduit. Treatment methods may include utilizing the stiffening member to advance the body portion of the device into position for the implantation.

In some examples, the device 100 may be implanted ab-internally (e.g., from inside the eye), such as through a clear corneal incision, and placed through the sclera and into a dissected subconjunctival space, as those of skill in the art will appreciate. In some other embodiments, the device 100 may be implantable ab-externally (e.g., from outside of the eye), such as through a conjunctival incision, as those of skill in the art should appreciate. In some embodiments, a conjunctival radial incision may be performed typically near the limbal junction, and blunt dissection of the conjunctiva may performed to expose the sclera and provide a site for placement of aqueous humor diffusion member. In some embodiments, this may require suturing of the aqueous humor diffusion member to the sclera. In some embodiments, a small needle, typically a 22-gauge or 23-gauge needle, may also be inserted near the scleral spur to provide a track for subsequent insertion and placement of the fluid conduit into the AC. Similarly, it will be appreciated that the various fluid conduit modifications discussed above may be performed through one or more of and ab-internal clear-corneal approach and an ab-external approach.

FIG. 1D is an exemplary image of a histopathology tissue slide showing the open collapsible body portion 102 and how the conjunctiva tissue of the eye interacts or engages with the body portion 102. In the image, the slide was stained with Masson's Trichrome (a histological stain agent) at 20× magnification (the bar at the lower right-hand corner indicates the scale showing 100 μm) such that collagen is stained blue and tissue cells are stained red. The body portion 102 includes the first surface 106 and the second surface 108, and the second surface 108 includes a plurality of ingrowth surface regions 118 and low-porosity regions 120. The ingrowth surface regions 118 allow tissue cells 122 from the conjunctiva to be received therein in order to facilitates ingrowth of the tissue cells 122 into a portion of the second surface 108, while the low-porosity regions 120 prevent or otherwise substantially inhibit such ingrowth therein. The first surface 106 is free or has an absence of any tissue cell or collagen, thereby indicating that although the second surface 108 may at least partially facilitate ingrowth of the tissue cells and collagen, the first surface 106 prevents or inhibits the ingrowth of the tissue cells 122 from external to the eye.

Additionally or alternatively, in various embodiments, the fluid conduits 104 of the various glaucoma drainage devices discussed here may be configured such that they include multiple lumens 114. FIG. 1E shows such example of the conduit 104 having four lumens 114A, 114B, 114C, and 114D through which fluid can enter (via the second end 112) and exit (via the first end 110) the conduit 104. In some embodiments, one or more of the lumens of the multilumen fluid conduit 104 can be initially blocked, and one or more of the lumens 114 (e.g., 114A through 114D) of the multilumen fluid conduit 104 can be post-operatively unblocked to increase a fluid flow rate through the fluid conduit 104. Thus, in various embodiments, the various glaucoma drainage devices discussed herein may include one or more mechanisms that can be post-operatively modified to increase and/or decrease the aqueous humor transmission rate per unit of time of the glaucoma drainage device. Accordingly, the glaucoma drainage devices discussed herein are operable to dynamically change to accommodate a change in the anatomy or functioning of the patient's eye post-op without requiring the need for further invasive surgery.

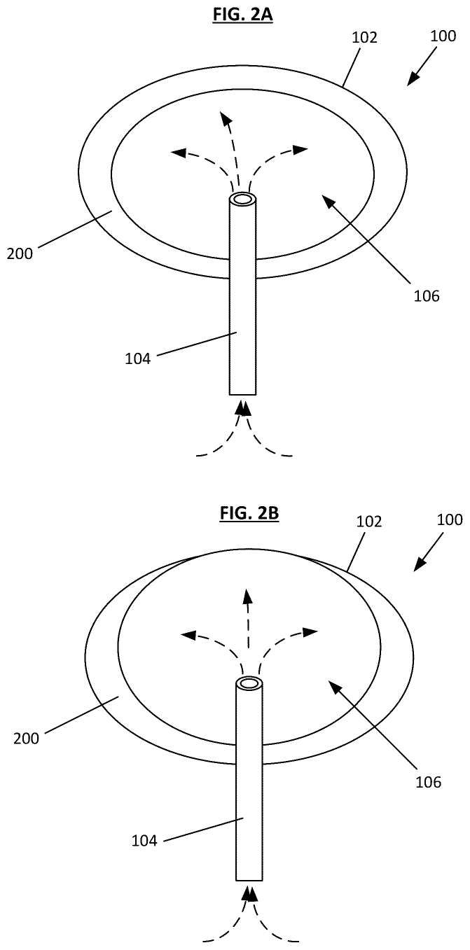

FIGS. 2A and 2B each shows an example of the drainage device 100 with a support structure 200 that is attached to at least a portion of the body portion 102. The support structure 200 is positioned at the first surface 106 on a periphery of the body portion 102. In some examples, the support structure 200 may cover at least 50%, at least 60%, at least 70%, at least 80%, at least 90%, or any other suitable range, combination of ranges, or value therebetween. In some examples, the support structure 200 may be a solid material, a solid and microporous material, a solid material with a microporous membrane attached thereto, or made of multiple components that are not directly attached to each other, as suitable and as further explained herein.

For example, in FIG. 2A, the support structure 200 covers all (or almost all) of the periphery to provide a more uniform stiffening effect on the body portion 102 of the device, while in FIG. 2B, a portion of the periphery at the top portion of the first surface 106 remains exposed to provide a differential stiffening effect on the body portion 102 where one end of the body portion 102 has a greater stiffness than the opposing end, as shown, with the stiffness gradually changing (that is, reducing or increasing) from one end to the other. In some examples, the conduit 104 may be attached to the support structure 200. In some examples, the support structure 200 may be made of any suitable biocompatible material that has a stiffness or rigidity greater than the body portion 102 of the device 100.

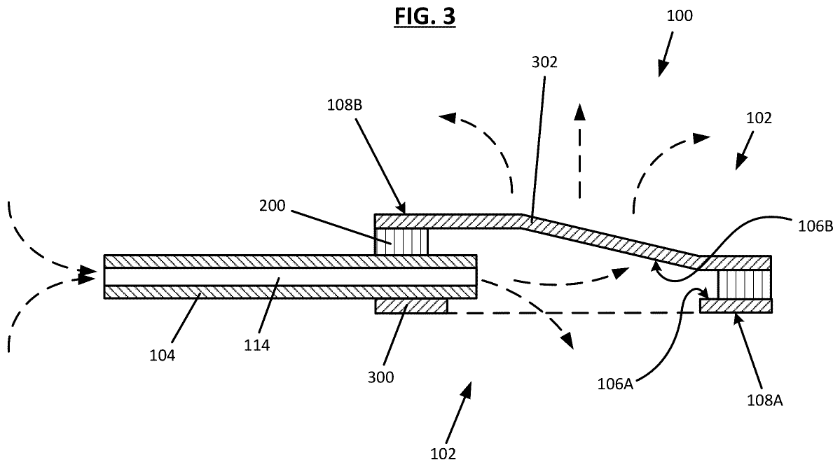

FIG. 3 shows an example of the drainage device 100 in which the body portion 102 includes an inner component 300 and an outer component 302. The inner and outer components may be two layers of membranes that are adhered, integrated, fused, and/or attached together using any suitable means. The word “inner” is used with respect to the eye in which the drainage device 100 may be implanted, such that an “inner” component is proximal to the eye while the “outer” component is distal to the eye relative to the inner component. As shown, the inner component 300 has a first surface 106A with a first surface porosity and a second surface 108A with a second surface porosity that is greater than the first surface porosity. The outer component 302 similarly has a first surface 106B and a second surface 108B. The first surface 106B has a first surface porosity and the second surface 108 has a second surface porosity that is greater than the first surface porosity. In some examples, the inner component 300 and the outer component 302 may be disposed such that the first surface 106A of the inner component 300 faces the first surface 1066 of the outer component 302.

In the example as shown, the conduit 104 may be disposed between the first surface 106A of the inner component 300 and the first surface 106B of the outer component 302. The support structure 200 may be disposed between the inner component 300 and the outer component 302. The first surfaces 106A and 106B may have similar surface porosities with respect to each other, and the second surfaces 108A and 108B may have similar surface porosities with respect to each other.

For example, the body portion 102 (or, in some examples, one or more of the inner component 300 and outer component 302 of the body portion 102) may include biocompatible materials such as expanded polytetrafluoroethylene (ePTFE). Additionally, one or more of the inner component 300 and outer component 302 may be formed of other biocompatible materials including biocompatible polymers, which may or may not be microporous, including, but not limited to, polyurethane, silicone, polysulfone, polyvinylidene fluorine (PVDF), polyhexafluoropropylene (PHFP), perfluoroalkoxy polymer (PFA), polyolefin, fluorinated ethylene propylene (FEP), acrylic copolymers, expanded polyethylene (ePE), and polytetrafluoroethylene (PTFE).

The inner component 300 and/or outer component 302 may be in the form of one or more sheets or films, and they may include knitted, woven, and/or non-woven forms including individual or multi-fiber strands. In some embodiments, the inner component 300 and/or outer component 302 may be formed from a plurality of sheets or films of polymer material. In some embodiments, the sheets or films may be laminated or otherwise mechanically coupled together to form the inner component 300 and/or outer component 302 as well as to form the body portion 102. Coupling of the sheets or films may be accomplished by a variety of mechanisms, including heat treatment, high pressure compression, bonding agents such as one or more adhesives, lamination, or other suitable methods known to one of skill in the art.

In some embodiments, adjacently-situated inner component 300 and outer component 302 and/or the layers of material forming such inner component 300 and outer component 302, may be partially or completely bonded via thermal methods (e.g., where one or both of the polymers forming the materials are brought to or above their melting temperature). In some embodiments, such thermal processes facilitate adhesive or cohesive bond formation between the materials or layers of material. In some embodiments, adjacently situated inner component 300 and outer component 302 and/or the layers of material forming such inner component 300 and outer component 302, may be partially bonded via thermal methods where at least one of the materials is brought to or above its melting temperature. Such thermal processes may facilitate adhesive or cohesive bond formation between the materials or layers of material. In some embodiments, one or more suitable adhesives are utilized and provide a sufficiently bonded interface. Adjacently situated inner component 300 and outer component 302 and/or the layers of material forming such inner component 300 and outer component 302 may be coupled together at one or more discrete locations (such as the periphery 700) to form stabilizing structures that extend through the resulting structure.

In some examples, the tube or conduit 104 and/or the support structure 200 may be formed of material or materials including but not limited to: PTFE, ePTFE, urethanes, polyurethane, silicones (organopolysiloxanes), polysulfone, PVDF, PHFP, PFA, polyolefin, FEP, ethylene fluorinated ethylene-propylene (EFEP), ethylene-tetrafluoroethylene (ETFE), and acrylic copolymers, among others. In some embodiments, the materials may include other biocompatible polymers suitable for use in forming the conduit 104 and/or the support structure 200 including, but not limited to, copolymers of silicon-urethane, styrene/isobutylene copolymers, polyisobutylene, polyethylene-co-poly(vinyl acetate), polyester copolymers, nylon copolymers, fluorinated hydrocarbon polymers and copolymers or mixtures of any of the foregoing may be used. In various embodiments, the elastomer or elastomeric material may include perfluorom ethyl vinyl ether and tetrafluoroethylene, (per)fluoroalkylvinylethers (PAVE), a copolymer of tetrafluoroethylene and perfluoromethyl vinyl ether, silicone, a fluoroelastomer, a urethane, butyl rubber, styrene-butadiene, isobutylene-isoprene, or a tetrafluoroethylene (TFE)/perfluoromethyl vinyl ether (PMVE) copolymer. In some examples, the support structure 200 may be formed using thermoplastics including, but are not limited to, acrylic and its copolymers, polyester and its copolymers, polypropylene, polystyrene, nylon and its copolymers, PTFE, among others.

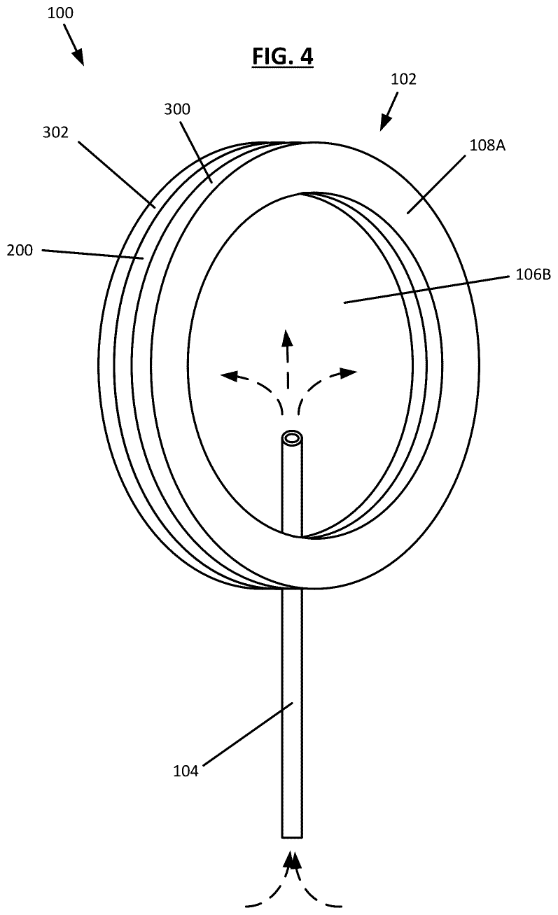

In FIG. 4, an example of the device 100 is shown such that the inner component 300 and the support structure 200 at least partially overlap with each other. The inner component 300 may be shaped like a washer or a flat ring, such that the center of the inner component 300 forms an opening through which fluid may exit after leaving the conduit 104. Similarly, the support structure 200 may resemble such washer but also includes an opening on its periphery to allow the conduit 104 to be inserted therethrough as shown, such that the conduit 104 can be disposed and affixed in position, surrounded by the support structure 200, the inner component 300, and the outer component 302. The second surface 108A of the inner component 300 and the first surface 1066 of the outer component 302 face the same direction as shown. Support structure 200 may be portions of the 300 and 302 that are heat-treated or sintered to weld or attach the two 300 and 302 together.

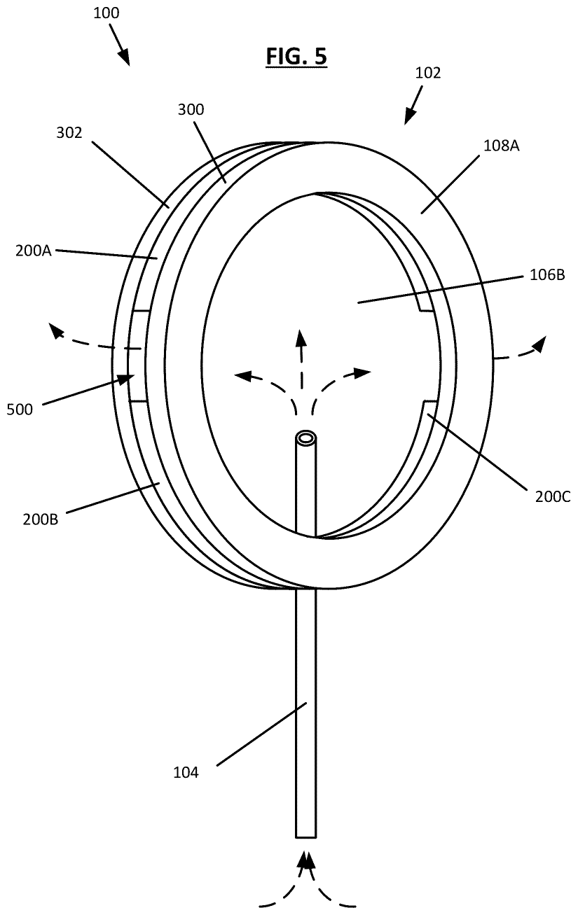

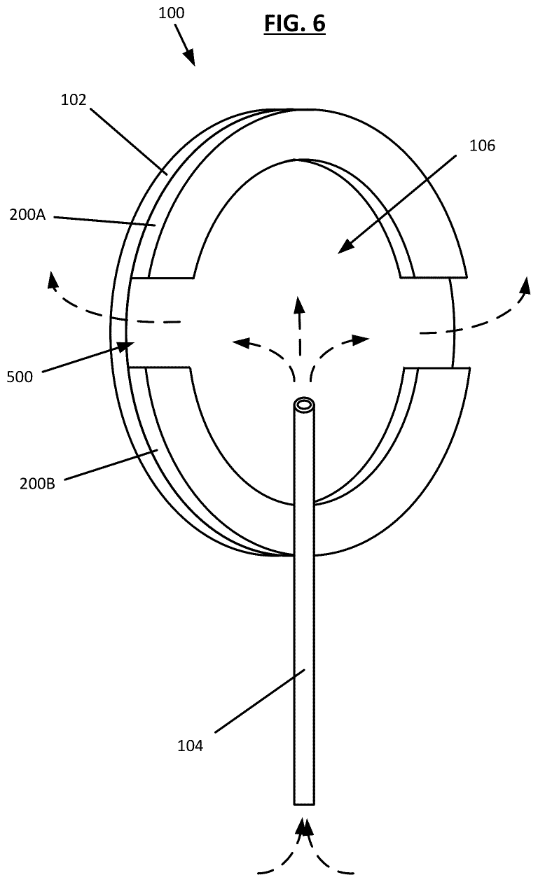

In FIG. 5, an example of the device 100 is shown where multiple support structure components 200A, 200B, and 200C are implemented. Although three support structure components are shown, there may be four or more components, or there may be two components (as shown in FIG. 6). In some examples, there may be two support structure components. In the example as shown, the conduit 104 is disposed between the support structure components 200B and 200C, while the support structure component 200A may be disposed such that one or more channels 500 are formed between the support structure component 200A and the other support structure components 200B and 200C. In some examples, the support structure components are disposed between the first surface 106A of the inner component 300 and the first surface 1066 of the outer component 302 in order to attach the first surface 106A to the first surface 106B, for example.

In some examples, fluid may leave from the channels 500, which may be defined by the support structure components 200A, 200B, and 200C as well as the inner component 300 and the outer component 302 of the body portion 102. In some examples, the support structure component 200A, 200B, or 200C may have the shape resembling an arch or semi-circle, or a quarter-circle, or any other suitable configuration based on how many support structure components there are. In some examples, where there are many smaller support structure components (for example, ten support structure components), each support structure component may be shaped like a square or rectangle and are disposed on the periphery of the body portion 102 such that multiple channels are formed therebetween. In some examples, each of the components of the support structure 200 may be made of a thermoplastic material.

FIG. 6 shows an example of the device 100 in which the support structure 200 has two support structure components 200A and 200B, where the conduit 104 is attached to the component 200B. In this example, the body portion 102 has a single component, so there are no inner and outer components as shown in FIG. 5. Instead, the channels 500 are defined by the body portion 102 (or more specifically, the first surface 106 of the body portion 102) and the support structure components 200A and 200B.

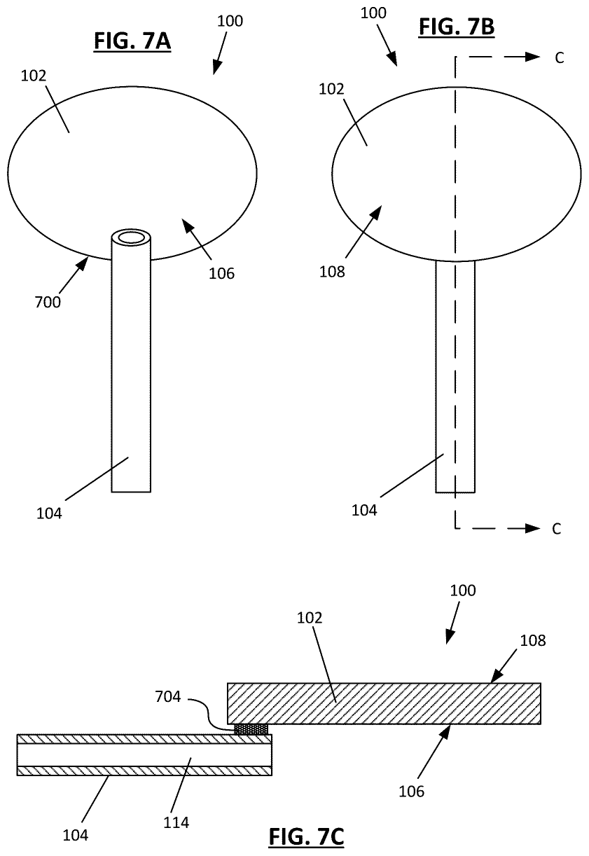

FIGS. 7A through 7C show an example of the device 100 in which the conduit 104 is attached to a periphery 700 of the first surface 106 of the body portion 102 using an adhesive 704 between the conduit 104 and the body portion 102. Any suitable adhesive may be used, including but not limited to heat-activated adhesive.

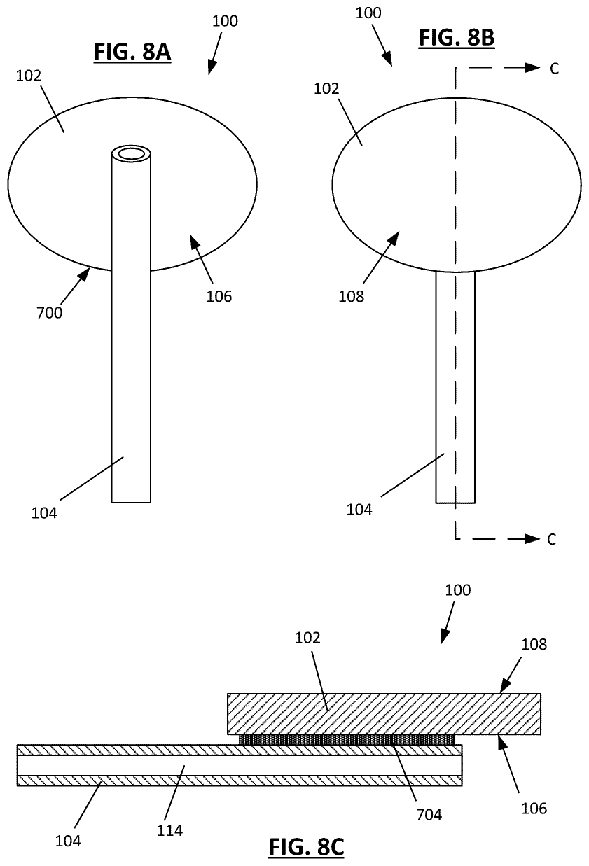

FIG. 8A through 8C show an example of the device 100 in which the conduit 104 is attached to the first surface 106 of the body portion 102 using the adhesive 704 which extends across at least 50% of a cross-sectional length of the body portion 102. In some examples, the conduit 104 may be attached to the body portion 102 across at least 50%, at least 60%, at least 70%, at least 80%, or any other suitable range, combination of ranges, or value therebetween.

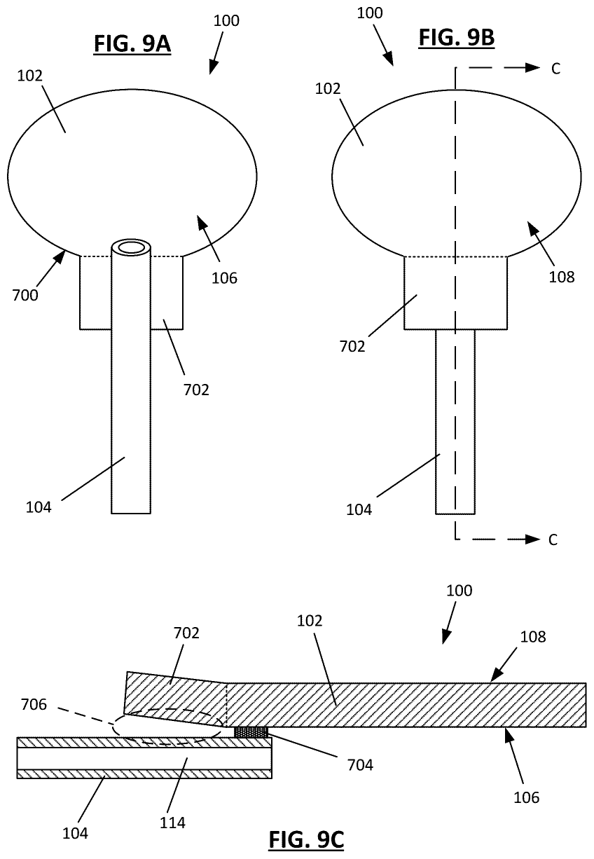

FIG. 9A through 9C show an example of the device 100 in which the conduit 104 is attached to the periphery 700 of the first surface 106 of the body portion 102 using the adhesive 704 between the conduit 104 and the body portion 102, and an erosion element 702 extends outwardly from the periphery 700 of the body portion 102 with respect to the location of the adhesive 704. The erosion element 702 remains unattached to the conduit 104 and is freely movable with respect to the conduit 104.

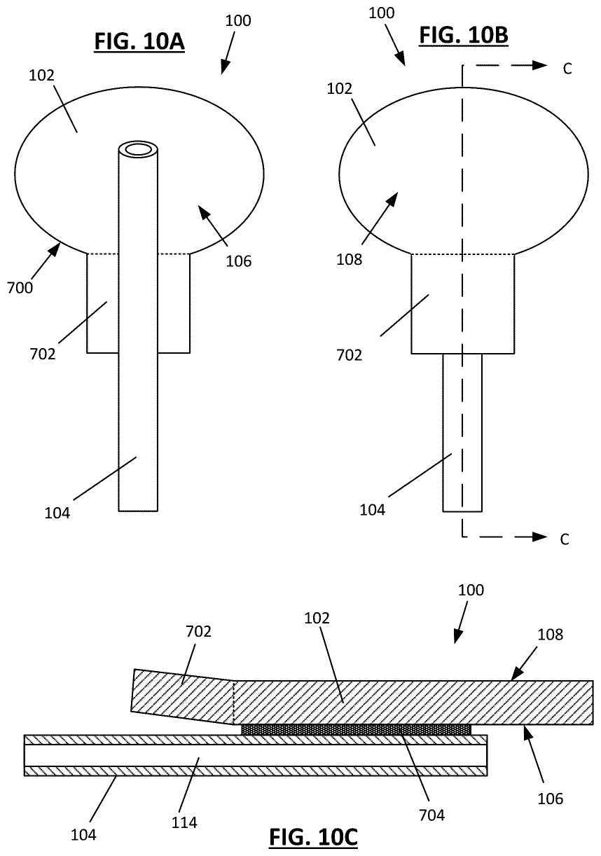

FIG. 10A through 10C show an example of the device 100 in which the erosion element 702 extends outwardly from the periphery 700 of the body portion 102 with respect to the location of the adhesive 704, and the conduit 104 is attached to the first surface 106 of the body portion 102 using the adhesive 704 which extends across at least 50% of a cross-sectional length of the body portion 102. In some examples, the conduit 104 may be attached to the body portion 102 across at least 50%, at least 60%, at least 70%, at least 80%, or any other suitable range, combination of ranges, or value therebetween. The erosion element 702 remains unattached to the conduit 104. In some examples, the erosion element 702 may be a separate component from the body portion 102, and the erosion element 702 may be affixed, attached, or adhered, temporarily or permanently to the body portion 102. In some examples, the erosion element 702 may be a continuous extension of the body portion 102.

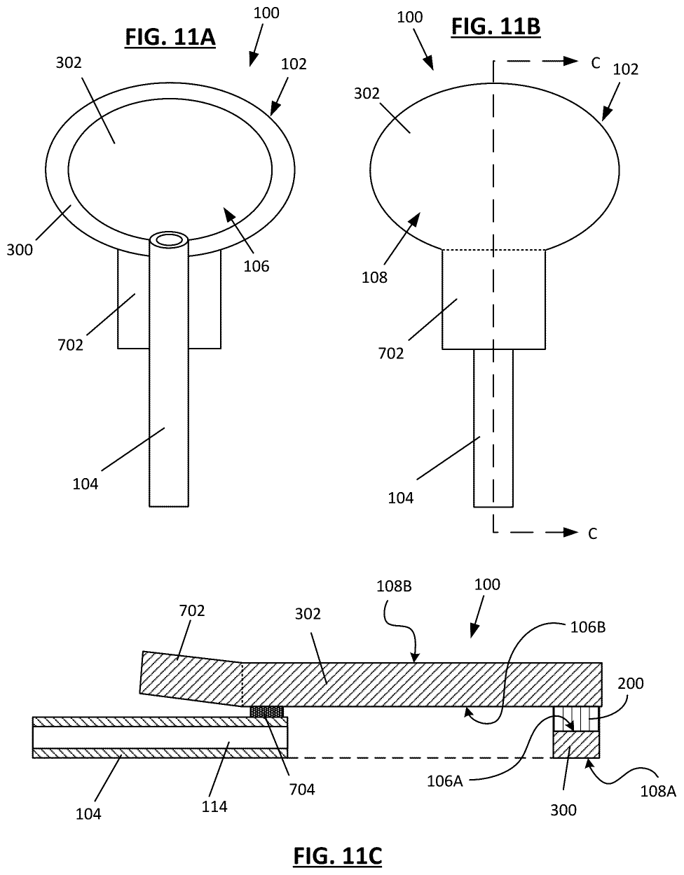

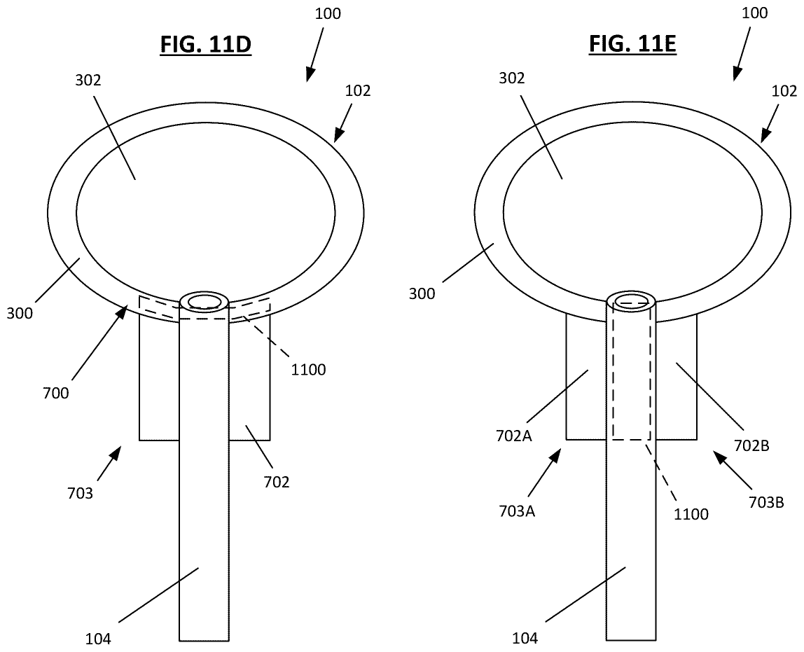

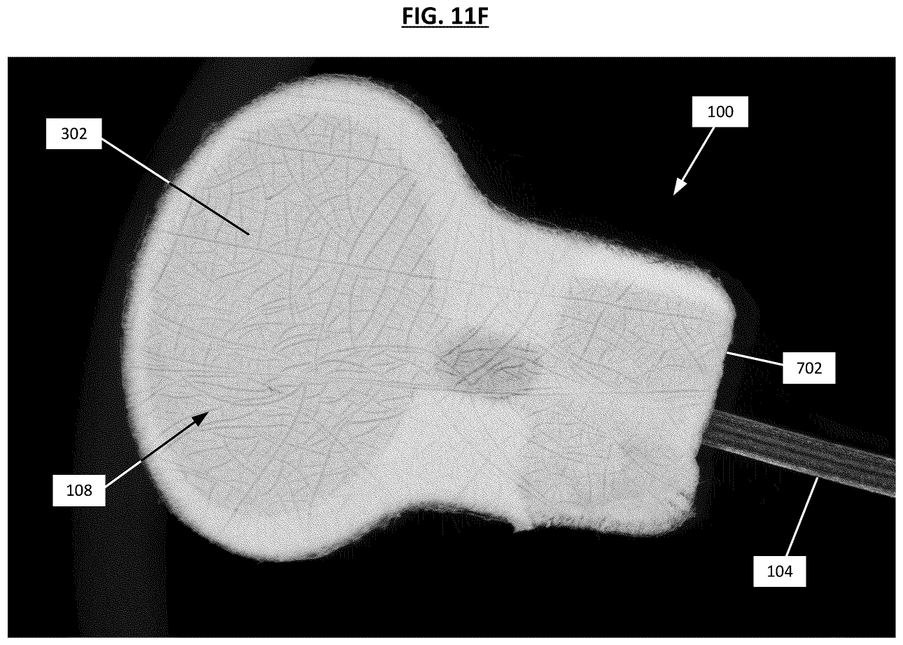

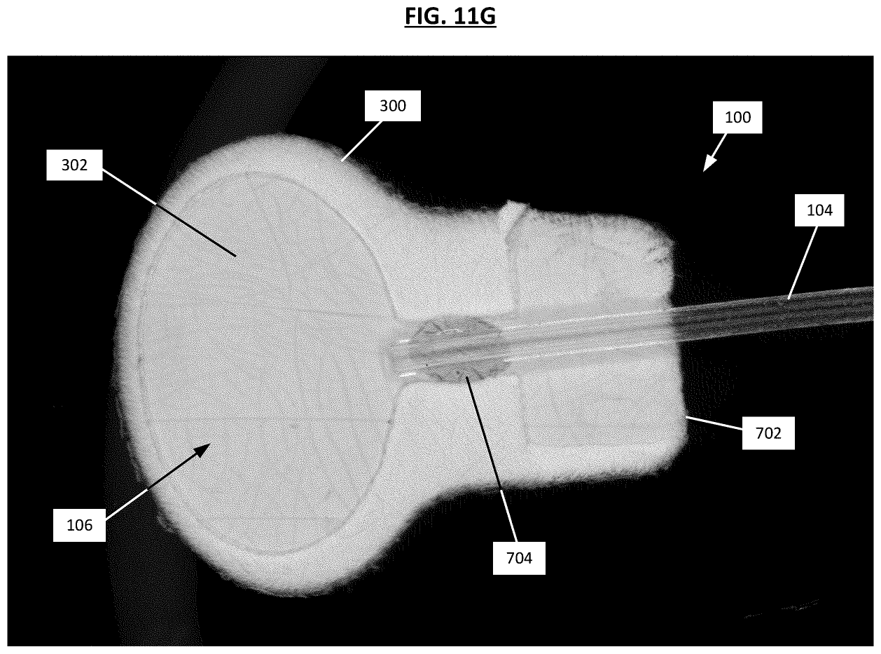

FIGS. 11A through 11G show examples of the device 100 in which the support structure 200 is disposed between the inner component 300 and the outer component 302 of the body portion 102. The adhesive 704 is disposed at an attachment region 1100 which may extend a length along the periphery 700 of the body portion 102 (as shown in FIG. 11D) or along a portion of the erosion element 702 (as shown in FIGS. 11E and 11G). In some examples, the attachment region 1100 may extend in both directions, i.e. both along the periphery 700 and along a portion of the erosion element 702.

When the attachment region 1100 extends along the portion of the erosion element 702, the erosion element 702 is separated into two portions: a first erosion element portion 702A and a second erosion element portion 702B located on either side of the attachment region 1100. Therefore, the erosion element 702 may be partially attached to the conduit 104 while the first portion 702A and the second portion 702B of the erosion element 702 remain unattached to the conduit 104. As such, these portions 702A and 702B are freely movable with respect to the conduit 104.

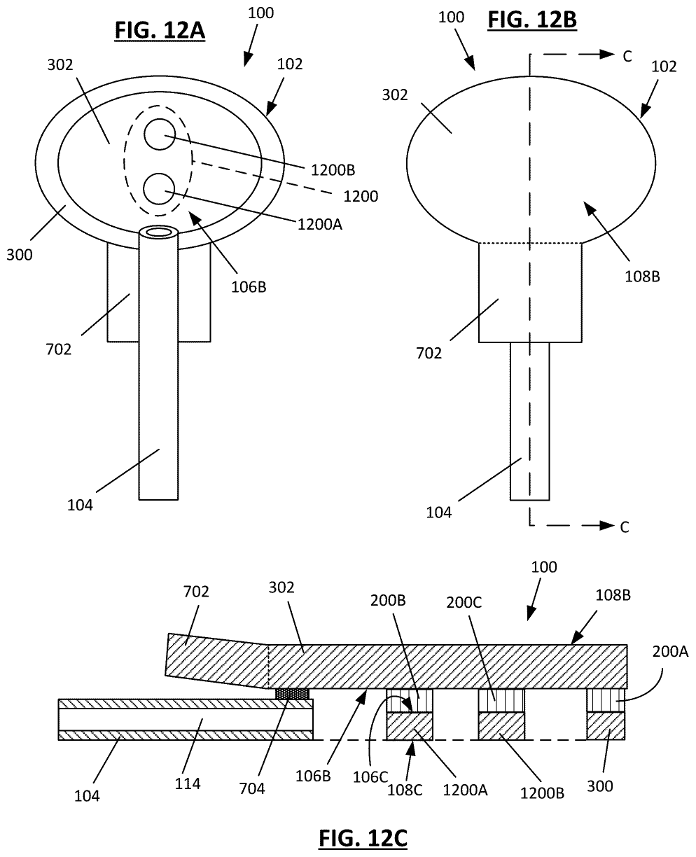

FIGS. 12A through 12C show an example of the device 100 in which, in addition to the body portion 102 having the inner component 300 and the outer component 302, the body portion 102 also includes one or more additional inner components 1200 that each has a first surface 106C with a first surface porosity and a second surface 108C with a second surface porosity that is greater than the first surface porosity. The additional inner components 1200 are attached to the outer component 302 such that each of the first surfaces 106C of the additional inner components 1200 faces the first surface 1066 of the outer component 302. As such, the second surface 1086 of the outer component 302 and the second surfaces 108C of the additional inner components 1200 face away from each other.

In some examples, there may be a single additional inner component. In some examples, there may be two or more additional inner components. In the example as shown, the device 100 includes two additional inner components 1200A and 1200B, and the support structure components 200A, 200B, and 200C are disposed such that the support structure component 200A is disposed between the inner component 300 and the outer component 302 of the body portion 102, the support structure component 200B is disposed between the additional inner component 1200A and the outer component 302 of the body portion 102, and the support structure component 200C is disposed between the additional inner component 1200B and the outer component 302 of the body portion 102. As shown, the support structure components 200B and 200C may be used to attach the first surfaces 106C of the additional inner components 1200A and 1200B, respectively, to the first surface 106B of the outer component 302 of the body portion 102. In some examples, each of the support structure components 200A, 200B, and 200C may be made of a thermoplastic material.

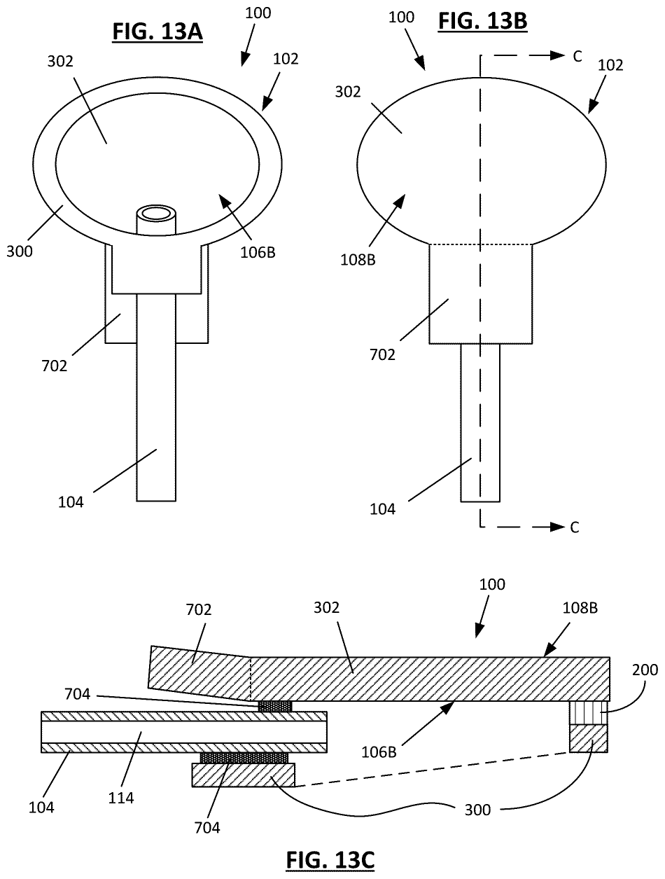

FIGS. 13A through 13C show an example of the device 100 in which a portion of the inner component 300 of the body portion 102 covers a portion of the conduit 104 such that the conduit 104 is at least partially disposed between the inner component 300 and the outer component 302. In some examples, the adhesive 704 is applied to both sides of the conduit 104 in order to attach the conduit 104 to both the inner component 300 and the outer component 302. In some examples, a portion of the inner component 300 may be attached to a portion of the erosion element 702 that extends from the outer component 302. The adhesive 704 applied may be a plurality of separate pieces of adhesive material, or the adhesive 704 may be a single piece of adhesive material that at least partially surrounds the outer surface of the conduit 104.

In various embodiments, an erosion element 702 is an element, feature, component, or portion of the device 100 that overlays a portion of the conduit 104 to help minimize erosion of the conduit 104 through one or more tissues of the eye when the device 100 is implanted. In various examples, the device 100 is implantable within a pocket formed between the conjunctiva and the sclera of the eye, as those of skill will appreciate.

In some instances, for example, the erosion element 702 extends from the body of the device 100 to overlay the conduit 104. The erosion element 702 operates as a protective barrier between the conduit 104 and one or more surrounding tissues of the eye. For example, the device 100 may be configured such that an erosion element 702 extends along the conduit 104 between the conduit 104 and a conjunctiva of the eye when implanted. In some such embodiments, the erosion element 702 helps minimize or even prevent erosion of the conduit 104 through the conjunctiva by forming a barrier between the conduit 104 and the conjunctiva when the device 100 is implanted in the eye.

In some embodiments, the erosion element 702 forms an integral, non-separable element, feature, component, or portion of the device 100. In some other embodiments, the erosion element 702 is formed as a distinct element or component that is coupled with one or more portions of the device 100. In some such embodiments, the erosion element 702 may be coupled with the one or more portions of the device 100. Alternatively, in some embodiments, the erosion element 702 may be coupled with the one or more portions of the device 100 such that the erosion element 702 can be subsequently separated and removed from the device 100.

In some such embodiments, the conduit 104 of the device 100 may be isolated from interfacing with the surrounding tissue of the eye (e.g., a sclera or a conjunctiva) by the incorporation of multiple erosion elements (702A and 702B, for example). That is, in some embodiments, the device 100 may include one or more erosion elements 702 that isolate the conduit 104 from the tissue of the eye. For instance, the device 100 may be configured such that erosion elements 702 flank the fluid conduit 104 on either side of a plane bisecting the fluid conduit 104 along a longitudinal axis thereof. In such a configuration, for example, a first one of the erosion elements 702 may extend along the fluid conduit 104 between the conduit 104 and a sclera of the eye. Similarly, a second one of the erosion elements 702 may extend along the fluid conduit 104 between the conduit 104 and a conjunctiva of the eye. Such a configuration provides erosion protection for both a conjunctiva and a sclera of an eye when the glaucoma drainage device 100 is implanted in the eye (e.g., when implanted within a pocket formed between the conjunctiva and the sclera), as the fluid conduit 104 is prevented from directly interfacing with the conjunctiva and the sclera of the eye.

In various embodiments, the erosion element 702 includes a thin, flexible, porous membrane consistent in construction, form, and makeup with the various other thin, flexible, porous mem brane(s) discussed herein. For example, the erosion element 702 may include a microstructure that is configured to resist tissue ingrowth (e.g., surface 106), or may alternatively include a microstructure that is configured to promote or permit tissue ingrowth (e.g., surface 108 such as in region 118 as shown in FIG. 1B). Alternatively, in some embodiments, the erosion element 702 may comprise a construct (which may be single-layered or multi-layered) including a first surface (or regions on a surface) configured to promote or permit tissue ingrowth (e.g., region 118 as shown in FIG. 1B) and a second surface (or regions on a surface) configured to resist tissue or cellular ingrowth (e.g., surface 106 as shown in FIG. 1B). The permissive/resistive layer(s) or region(s) in such embodiments are oriented to optimize their effect when the glaucoma drainage device 100 is implanted in the eye. For instance, as discussed in greater detail below, in various embodiments, the erosion element 702 is configured to promote or permit tissue ingrowth along an interface between the erosion element 702 and a tissue of the eye (e.g., such as the sclera or the conjunctiva). It will thus be appreciated that the material of the erosion element 702 may include any material and may be constructed according to any method discussed herein as being suitable for the layer(s) or region(s) as discussed.

Accordingly, in various embodiments, the erosion element 702 may be coupled with (or alternatively may be an extension of or integral with) any of the components of the body portion 102 as discussed herein. Thus, in some embodiments, the erosion element 702 may itself be a membrane or construct with a porosity that is configured to minimize, resist, or prevent tissue ingrowth, or a membrane or construct with a porosity that is configured to permit tissue ingrowth.

In some examples, the erosion element 702 extends away from the body portion 102 of the device 100 as shown. In some embodiments, the erosion element 702 extends away from the outer component 302 of the body portion 102 along the fluid conduit 104 between the conduit 104 and the inner component 300 of the body portion 102. In some embodiments, the erosion element 702 extends between the inner component 300 of the body portion 102 and an end of the fluid conduit 104 (e.g., a first end or a second end of the fluid conduit 104) that is configured to access a biological fluid-filled body cavity, such as an anterior chamber of an eye, among other embodiments as will be appreciated by those of skill in the art.

Though illustrated in FIGS. 9A through 9C, 10A through 10C, 11A through 11G, 12A through 12C, 13A through 13C, and 14A through 14C as including a rectangular shape, it will be appreciated that the erosion element 702 may be of any suitable shape without departing from the spirit or scope of the disclosure. For instance, the erosion element 702 may be square, rectangular, trapezoidal, or some other polygonal shape, and may include chamfered or rounded edges between sides, and the sides may be linear or generally curved in nature. The erosion element 702 may have a generally continuous curved edge in that it is circular or ovular, or of another suitable shape (e.g., bean-shaped). It is to be appreciated that one of skill in the art will appreciate that the erosion element 702 may be of any desired shape provided that the erosion element 702 helps protect against erosion of the fluid conduit through tissue surrounding the fluid conduit and provided the erosion element 702 can be placed within a subconjunctival space (such as a pocket formed between the conjunctiva and the sclera) as described herein.

In some embodiments, the erosion element 702 extends along a length of the fluid conduit, but includes a length that is shorter than a length of the portion of the fluid conduit extending from the body portion 102. In other embodiments, the erosion element 702 extends along a length of the fluid conduit, and includes a length that is equal to or greater than a length of the portion of the fluid conduit extending from the body portion 102. In some embodiments, the erosion element 702 has a width that is greater than or equal to a diameter of the fluid conduit 104. However, in some embodiments, the width of the erosion element 702 may be less than the diameter of the fluid conduit, provided that the erosion element 702 is not rendered ineffective against helping protect against erosion of the fluid conduit through surrounding tissue. Consistent with the versatility in suitable sizes and shapes of the erosion element 702 discussed above, it will be appreciated that the width of the erosion element 702 may remain constant along the length of the erosion element 702, or alternatively, the width of the erosion element 702 may vary along the length of the erosion element 702. For example, the width may taper (linearly or nonlinearly) along the longitudinal length of the erosion element.

In some embodiments, the erosion element 702 may be configured such that it is more abrasion resistant in high wear or high abrasion areas (e.g., areas where the fluid conduit 104 has a potential to move relative to the erosion element 702). Resistance to abrasion in such areas may be accomplished according to any known methods, including material compositions and/or material thickness. A thickness of the erosion element 702 may thus vary along the length of the erosion element 702, and/or may vary laterally across its width. For example, the thickness may taper (linearly or nonlinearly) along the length of the erosion element 702 and/or transversely thereacross. For instance, a thickness of the erosion element 702 along a longitudinally extending centerline may be in excess of a thickness of the erosion element 702 along one or more of its longitudinally extending edges. Alternatively, it will be appreciated that a thickness of the erosion element 702 along a longitudinally extending centerline may be less than a thickness of the erosion element 702 along one or more of its longitudinally extending edges. Additionally or alternatively, a thickness of the erosion element 702 along a section of its longitudinal length may be in excess of a thickness of the erosion element 702 along a second section of its longitudinal length. For example, if a region where the fluid conduit 104 accesses the fluid-filled body cavity corresponds to a high abrasion region, a section of the erosion element 702 that is more proximate the end of the fluid conduit 104 that is configured to access the fluid-filled body cavity may be thicker than is a section of the erosion element 702 that is more proximate the body portion 102. It is to be appreciated that a thickness of the erosion element 702 can be optimized in high wear or high abrasion areas to reduce a risk of premature failure of the device 100, due to abrasion of the erosion element 702 by the fluid conduit 104. These variances in thickness may be achieved through selective layering of materials that collectively form the erosion element 702 or other known methods.

In some embodiments, the erosion element 702 may be longitudinally spaced apart from the body portion 102, or may include a region of reduced width (not illustrated) and/or thickness (not illustrated) extending between the erosion element 702 and the body portion 102 along those regions of the fluid conduit 104 that are associated with a low risk of erosion through the surrounding tissue. For example, if the portion of the fluid conduit 104 adjacent the body portion 102 is associated with a low risk of erosion through the surrounding tissue, a region of reduced width and/or thickness of the erosion element 702 may be situated adjacent this region of the fluid conduit 104. Alternatively, the erosion element 702 may be configured such that the fluid conduit 104 is exposed to the surrounding tissue in this region of low risk for erosion. Thus, in some examples, the erosion element 702 may not extend from the body portion 102.

In some embodiments, the erosion element 702 is coupled to the fluid conduit 104. The erosion element 702 may be coupled to the fluid conduit 104 continuously along a length of the fluid conduit 104, or alternatively along the fluid conduit 104 at one or more discrete locations. The erosion element 702 may be coupled to the fluid conduit 104 according to any known methods including, but not limited to suturing or stitching of the erosion element along the length of the conduit. In some embodiments, suturing can be a series of interrupted sutures or a continuous running stitch. Additionally or alternatively, the fluid conduit 104 can be mechanically adhered to the erosion element 702 by partially melting the fluid conduit 104 into the microporous structure of the erosion element 702. In some embodiments, the erosion element 702 may be coated with an adhesive that is tacky such that the fluid conduit 104 can releasably stick to the erosion element 702. In some embodiments, one or more bands of material (e.g., microporous material) can have their ends adhered to the erosion element 702 such that an eyelet is formed between the band of material and the erosion element 702 and the fluid conduit 104 can be threaded through the gap. In some examples, as shown in FIG. 11D, a trimmable portion 703 of the erosion element 702 may be trimmed (e.g., via cutting) by the physician so as to adjust the length or area of the erosion element 702 by removing portion(s) thereof that is not required for the procedure. In some examples, as shown in FIG. 11E, there may be a plurality of trimmable portions 703A and 703B, such as when there are multiple erosion elements (e.g., 702A and 702B as shown).

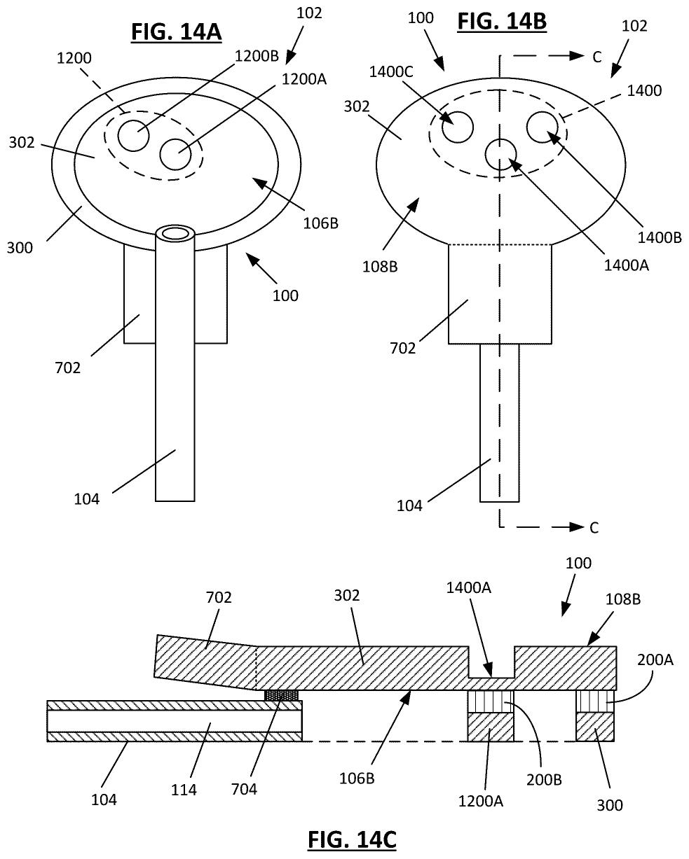

FIGS. 14A through 14B show an example of the device 100 in which the body portion 102 includes one or more third surfaces 1400. A third surface 1400 may be defined as a region in the second surface 108 (which, in the example shown, corresponds to the second surface 108B of the outer component 302 of the body portion 102) that opposes the first surface 106 (which, in the example shown, corresponds to the first surface 106B of the outer component 302 of the body portion 102). The third surface 1400 is discontinuous from the second surface 108 and has a third porosity that is less than the second porosity of the second surface 108, similar to the low-porosity region 120 as described elsewhere herein. In some examples, the third porosity of the third surface 1400 equals the first porosity of the first surface 106.

In some examples, the third surfaces 1400 may be depressions formed on the second surface 108 such as that shown in FIG. 14C, where the depressions are formed by removing a portion of the body portion 102 (or, in the example shown, a portion of the outer component 302 of the body portion 102) with the second (higher) porosity. According to some examples, the third surfaces 1400 may be depressions formed by applying a pressure or pressures to reduce the thickness of the body portion 102 (or, in the example as shown, the outer component 302 of the body portion 102).

In the example as shown, there are three third surfaces 1400A, 1400B, and 1400C and two additional inner components 1200A and 1200B. As such, the location of these third surfaces and additional inner components may be determined such that each third surface 1400 may be located on the opposite side of the outer component 302 of the body portion 102 from one of the additional inner components 1200 (e.g., the third surface 1400A may be paired with the additional inner component 1200A, and the third surface 1400B may be paired with the additional inner component 1200B). In some examples, there may be a third surface that is not paired with any additional inner component, such as the third surface 1400C.

Advantages of implementing the embodiments of the drainage device as disclosed herein include allowing the formation of a significantly thinner conjunctival capsule relative to the solid silicone plate “tube shunt” incumbents of the Ahmed glaucoma valve as known in the art (e.g., refer to the prior-art example shown in FIGS. 15A through 15C), and implementing the open collapsible body portion as disclosed herein facilitates alleviating the stress that is applied on the conjunctiva by the device when implanted, thereby minimizing the stress that may be experienced by the eye when subjected to the pressure from the AC. The thinness may provide benefits including, but are not limited to: requiring less material to produce, having a simpler design to make and modify, having a lighter weight, being more low-profile, being minimally invasive when implanted, and adding longevity to the device because the lack of any chamber or reservoir ensures that the device would not fill up with cells and cellular debris after extended use.

Furthermore, an interior of the body portion with sufficient microporosity defines a surface area for the device through which aqueous flux (fluid flow) to occur. Also, one exterior surface of the device may have a sufficiently greater microporosity to facilitate tissue ingrowth, but an opposing exterior surface of the body portion may have a sufficiently lesser microporosity in order to prevent the tissue ingrowth to reach the opposing exterior surface, thereby allowing an appropriate amount of tissue ingrowth to occur on one surface of the device while simultaneously reducing the problem of having too much tissue ingrowth occurring on both surfaces of the device, and allowing fluid to flow from one surface (e.g., from the eye) to another surface (e.g., into the conjunctiva) as suitable. Furthermore, the conduit (tube) is protected from the conjunctiva by positioning the conduit on the scleral side of the body portion as described. Such protection may be beneficial in reducing the risk of the conduit being clogged at the distal (conjunctival) end of the device, for example. Additionally, stiffening element(s) such as the support structure (or components thereof) positioned at the periphery of the device can aid in the handling of the device for implantation by the practitioner (e.g., doctor, surgeon, or physician) while minimizing the risk of the practitioner accidentally folding the body portion of the device in vivo, which may negatively compromise the efficacy of the device or render the device useless.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

What is claimed is:

1. A drainage device for draining a biological fluid from an eye to a tissue external to the eye, the drainage device being implantable at least in part within a tissue of the eye and comprising:
an open collapsible body portion having a first surface with a first porosity and a second surface with a second porosity that is greater than the first porosity, the second surface opposing the first surface; and
a conduit having a first end fluidly coupled with the first surface and a second end insertable into the eye to facilitate a drainage of the biological fluid into the conduit,
wherein the second surface with the second porosity includes at least one ingrowth surface region for facilitating ingrowth of tissue external to the eye.

2. The drainage device of claim 1, wherein the first surface with the first porosity is configured to inhibit the ingrowth of the tissue external to the eye.

3. The drainage device of claim 1, wherein the second surface further includes at least one low-porosity region for inhibiting the ingrowth of the tissue external to the eye.

4. The drainage device of claim 3, wherein the at least one low-porosity region in the second surface defines a third surface opposing the first surface that is discontinuous from the second surface and has a third porosity that is less than the second porosity.

5. The drainage device of claim 4, wherein the third porosity is equal to the first porosity.

6. The drainage device of claim 1, wherein the body portion comprises a plurality of internal regions with porosities that are greater than the first porosity and less than the second porosity.

7. The drainage device of claim 1, wherein the conduit is attached to a periphery of the body portion.

8. The drainage device of claim 1, wherein the conduit is attached to the body portion across at least 50% of a cross-sectional length of the body portion.

9. The drainage device of claim 1, wherein the conduit is attached to the first surface of the body portion at an attachment region, and the body portion further comprises an erosion element with a trimmable portion that extends outwardly from the attachment region with respect to the conduit.

10. The drainage device of claim 9, wherein the erosion element is a separate component from the body portion.

11. The drainage device of claim 9, wherein the erosion element is a continuous extension of the body portion.

12. The drainage device of claim 1, the body portion comprising:
an inner component having a first surface with a first surface porosity and a second surface with a second surface porosity that is greater than the first surface porosity, and
an outer component having a first surface with a first surface porosity and a second surface with a second surface porosity that is greater than the first surface porosity,
wherein the inner and outer components are disposed such that the first surface of the inner component faces the first surface of the outer component.

13. The drainage device of claim 12, wherein the conduit is disposed between the first surface of the inner component and the first surface of the outer component.

14. The drainage device of claim 12, further comprising:
one or more additional inner components each having a first surface with a first surface porosity and a second surface with a second surface porosity that is greater than the first surface porosity,
wherein the additional inner components are attached to the outer component such that each of the first surfaces of the additional inner components faces the first surface of the outer component.

15. The drainage device of claim 1, further comprising a support structure attached to at least a portion of the body portion.

16. The drainage device of claim 15, wherein the conduit is attached to a portion of the support structure.

17. The drainage device of claim 15, wherein the support structure comprises one or m ore thermoplastic components.

18. The drainage device of claim 17, wherein one or more channels are formed between two of the thermoplastic components through which the biological fluid is configured to pass.

19. The drainage device of claim 12, further comprising a support structure attached to at least a portion of the body portion, wherein the support structure is disposed between the first surface of the inner component and the first surface of the outer component.
